(19) United States
(12) Patent Application Publication
(10) Pub. No.: US 2007/0298496 A1
(43) Pub. Date: Dec. 27, 2007

(54) Method of deriving pluripotent stem cells from a single blastomere

(76) Inventors: Hung-Chih Kuo, Taipei (TW); Ya-Ling Chen, Husi Town (TW); Yu-Ting Yan, Taipei (TW); Chia-Ning Shen, Taipei (TW); Shu-Hwa Chen, Taipei (TW); John Yu, Taipei (TW); Ching-Yu Chuang, Taipei (TW)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/651,563

(22) Filed: Jan. 10, 2007

Related U.S. Application Data
(60) Provisional application No. 60/815,842, filed on Jun. 23, 2006.

(51) Int. Cl. C12N 5/06

(52) U.S. Cl. 435/377; 435/325

(57) ABSTRACT

The present invention provides a high efficiency method of deriving pluripotent mammalian stem cells from single, dissociated blastomeres harvested from preimplantation embryos.

### CROSS REFERENCE TO RELATED APPLICATIONS

The present invention is related to and claims the benefit of, under U.S.C. §119(e), U.S. provisional patent application Ser. No. 60/815,842, filed on Jun. 23, 2006, which is expressly incorporated fully herein by reference.

### FIELD OF THE INVENTION

This invention relates to methods of deriving pluripotent mammalian stem cells.

### BACKGROUND OF THE INVENTION

Embryonic stem cells (ESC) are self-renewing, pluripotent, and have the capacity to give rise to all of the tissue types of the body. Accordingly, the elucidation of the cellular and molecular mechanisms that control pluripotency and self-renewal in ESC could lead to enhanced treatment of a wide range of human conditions that can be attributed to the loss or malfunction of specific cell types. In addition, such knowledge would also contribute to the understanding of mechanisms underlying cell differentiation, cell self-renewal, and mechanisms related to early development. Consequently, researchers have attempted to isolate and propagate ESC from developing mammalian embryos. To this end, ESC that maintain pluripotency during storage and in vitro and in vivo culturing, have been successfully derived from embryos of various species (Evans M J, et. al. (1981) Nature 292:154-156), including non-human primates (Suemori H, et. al. (2001) Dev Dyn 222:273-279; Thomson J A, et. al. (1995) Proc Natl Acad Sci 92:7844-7848) and humans (Reubinoff B E, et. al. (2000) Nat Biotechnol 18:399-404; Thomson J A, et. al. (1998) Science 282:1145-1147). Established methods of deriving mammalian pluripotent ESC include using whole blastocysts or their associated inner cell mass (ICM) as the starting material. However, these methods result in the destruction of the developing embryo, a step that has triggered ethical concerns with regard to human embryos.

As a possible alternative to the isolation of pluripotent stem cells from the ICM, a number of researchers have attempted to derive pluripotent stem cells from preimplantation embryos. This approach has been viewed favorably, in part because preimplantation embryos are capable of developing normally following the removal of a blastomere (Hardy K, et al. (1996) Mol Hum Reprod 2:621-32; Handyside A H, et al. (1990) Nature 344:768-70; Hardy K, et al. (1990) Hum Reprod 5:708-14; Sermon K D, et al. (2006) Verh K Acad Geneeskd Belg 68:5-32; Matsumoto K, et al. (989) Gamete Res 22:257-63; Papaioannou V E, et al. (989) Development 106:817-27; Saito S, et al. (1991) Biol Reprod 44:927-36; Allen W R, et al. (984) J Reprod Fertil 71:607-13; Chan A W, et al. Science 287:317-19; Mitalipov S M, et al. (2002) Biol Reprod 66:1449-55.). Indeed, studies have reported the derivation of ESC-like cells from blastomeres isolated from preimplantation embryos (Strelchenko N, et. al. (2004) Reprod Biomed Online 9:623-629; Tesar P J, et. al. (2005) Proc Natl Acad Sci 102:8239-8244) (Tesar P J, et. al. (2005) Proc Nati Acad Sci 102:8239-8244; Delhaise F, et. al. (1996) Eur J Morphol 34:237-243). However, these particular methods also resulted in the necessary destruction of the donor embryo.

Most of the attempts at producing ESC-like pluriopotent stem cells from preimplantation embryos involved embryos at the 2 cell, 4 cell, and 8 cell stages of embryonic development. Single blastomeres isolated from embryos at these stages are conventionally referred to as ½, ¼, and ⅛ blastomeres, respectively. In particular, ⅛ blastomeres from non-human primates, humans, and other mammalian species can give rise to pluripotential stem cells comparable to ESC when the blastomeres are cultured in groups of either 2 or 4 (U.S. patent publication 2003/0106082 A1). ESC lines were also generated from blastomeres by co-culturing the blastomeres with conventionally produced ESC lines (Chung Y et al. (2006) Nature 439:216-219). Single blastomeres isolated from pre-implantation embryos were also used to produce blastocyst-like structures with visible ICM structures, although the number of ICM cells was significantly less than that observed for developmental-stage-matched control embryos. However, higher numbers of ICM cells were derived from single rabbit blastomeres by supplementing the culture medium with serum (Tao T and Niemann H (2000) Human Reproduction 15:881-89). Preimplantation mouse blastomeres can also contribute to the development of normal pups. For example, ⅛ blastomeres or pairs of blastomeres isolated from 16 cell embryos were aggregated with 4-cell embryos that had been made tetraploid, and implanted into surrogate mothers which resulted in the birth of normal pups fully derived from the donor blastomeres (Tarkowski A K et al. (2005) Int J Dev Biol 49:825-32). Similarly, non-human primate offspring can be derived from a pair of blastomeres that were isolated from an 8 cell embryo, inserted back into the empty zona pellucida, and implanted into a surrogate mother (Chan A W S et al. (2000) Science 287:317-319). Such an observation indicates that a pair of blastomeres can give rise to embryonic and extra-embryonic tissue, meaning that blastomeres may actually be able to be characterized as “totipotent” stem cells. To date though, there are no reports of the generation of pluripotent stem cells from single, isolated blastomeres without the aid of co-culture with other stem cells or blastomeres.

The ability to derive pluripotent stem cells from single, isolated blastomeres would avoid many of the ethical concerns associated with the destruction of embryos. However, as discussed above, known methods of excising and culturing single blastomeres have failed to produce stem cells that demonstrated the pluripotency normally associated with ESC (Eckert J, et al. (1997) Biol Reprod 57:552-60; Rossant J. (976) J Embryol Exp Morphol 36:283-90; Tarkowski A K. (959) Nature 184:1286-87).

A method of deriving pluripotent stem cells that avoids the ethical concerns associated with ESC is desirable and would likely facilitate the implementation of stem-cell based therapies.

### SUMMARY OF THE INVENTION

The present invention pertains to a method of deriving pluripotent stem cells from single, isolated blastomeres of preimplantation mammalian embryos. In one embodiment, the pluripotent stem cells of the invention express cell markers that are known in the art to be useful for identifying ESC.

The blastomeres used to derive pluripotent stem cells are isolated from embryos at either the two-cell, four-cell, or eight-cell stage of embryonic development. Known differential growth requirements for different preimplantation stages of development are considered for the determination of blastomere culture conditions (Gardner D K. (1998) Theriogenology 49:83-102; Gardner D K, et al (988) Development 104:423-29; Hardy K, et al. (989) Hum Reprod 4:188-91). In one embodiment of the invention, single, isolated blastomeres are cultured in KSOM EmbryoMax® medium (Specialty Media, Phillipsburg, N.J., Phillipsburg, N.J.). Each blastomere is cultured in at least 5 μl and as much as 500 μl of culture medium, but preferably in 10 μl to 50 μl of culture medium. In some species, the glycoprotein leukemia inhibitory factor (LIF) effectively maintains pluripotency of ESC (Nichols J, et al. (1990) Development 110:1341-48) and also serves a function in blastocyst development (Cheng T C, et al. (2004) Biol Reprod 70:1270-76; Dunglison G F, wt al. (1996) Hum Reprod 11:191-96; Lavranos T C, et al. (1995) J Reprod Fertil 105:331-38; Stewart C L, et al. (1992) Nature 359:76-9. In one embodiment the culture medium additionally comprises between 10 and 10⁴ I.U. of LIF per ml, preferably between 500 and 1500 I.U. per ml.

Morula-like cell clusters form within one to two days of culture under the conditions described above, which generally is 2.5 to 3 days post-coitus (p.c.) for mice. The morula-like cell clusters that form under the culture conditions described above are transferred to a co-culture system, wherein the morula-like cell clusters are co-cultured on a monolayer of mitomycin C-inactivated mouse embryo fibroblasts (MEF) that are plated on gelatin coated tissue culture plates. In one embodiment, the co-culture system of the invention comprises ESC culture medium (80% Dulbecco's modified Eagle medium supplemented with 20% fetal bovine serum, 0.1 mM β-mercaptoethanol, 1% nonessential amino acids, and 2 mM glutamine). In another embodiment, the co-culture system of the invention comprises ESC culture medium supplemented with between 10 and 10⁴ I.U. of LIF per ml, preferably between 500 and 1500 I.U. per ml.

The blastomere-derived cells are cultured in the co-culture system of the invention until clusters of cells with ESC-like morphological traits, such as a high ratio of nucleus to cytoplasm and prominent nucleoli, form dome-shaped colonies of cells. Generally, such colonies are apparent within about two to three days of culturing. In one embodiment, the blastomere-derived cells are passaged by mechanically selecting colonies of cells with ESC-like morphology from the co-culture system of the invention, dissociating the colonies into single cell suspensions, and introducing the cells to a fresh MEF co-culture system as described above. In another embodiment, ESC-like cells are selected for passaging by Fluorescence Activated Cell Sorting (FACS) by using a fluorescently-tagged antibody that is specific for markers of pluripotent stem cells, such as an antibody specific for stage-specific mouse embryonic antigen (SSEA-1). Blastomere-derived ESC-like cells are passaged through the co-culture system from 0 to 100 times, but preferably from 1 to 20 times. Blastomere-derived pluripotent stem cells are passaged through the co-culture system until enough cells are available for characterization, analysis, and cryopreservation. It is preferable that there be at least 1000 cells available for characterization and preservation. More preferably though, would be to have at least 2×10³ cells available for characterization and cryopreservation.

The blastomere-derived pluripotent stem cells of the invention express markers commonly used to identify self-renewing pluripotent ESC. The polynucleotides of ESC markers can be detected by any means of polynucleotide detection methods known to one of ordinary skill in the art, including, but not limited to reverse-transcript polymerase chain reaction (RT-PCR), real-time PCR, and Northern blot analysis. Likewise, polypeptides of ESC markers can be detected by any means of polypeptide detection method known to one of ordinary skill in the art, including, but not limited to immunohistochemical methods, FACS analysis, and Western blot analysis. In one embodiment, the blastomere-derived pluripotent stem cells of the invention express the POU family transcription factor, Octamer 4 (Oct-4). In another embodiment, the blastomere-derived pluripotent stem cells of the invention express SSEA-1. In another embodiment, the blastomere-derived pluripotent stem cells of the invention express Nanog. In another embodiment, the blastomere-derived pluripotent stem cells of the invention express Rex-1, which is also referred to as zinc-finger protein-42 (Zfp42). In another embodiment, the blastomere-derived pluripotent stem cells of the invention express alkaline phosphatase. In another embodiment, the blastomere-derived pluripotent stem cells of the invention express FOXD3. In another embodiment, the blastomere-derived pluripotent stem cells of the invention express undifferentiated embryonic cell transcription factor (UTF)-1. In another embodiment, the blastomere-derived pluripotent stem cells of the invention may express more than one of any of the ESC markers provided above. The blastomere-derived pluripotent stem cells of the invention also have normal euploid karyotypes that can be maintained after periods of in vitro culture lasting more than six months.

The blastomere-derived pluripotent stem cells of the invention are able to give rise to somatic cell types that represent the endodermal, ectodermal, and mesodermal embryonic germ layers. In one embodiment, blastomere-derived pluripotent stem cells of the invention can produce teratomas that comprise endodermal, ectodermal, and mesodermal cell types. In another embodiment, blastomere-derived pluripotent stem cells of the invention can produce embryoid bodies in vitro comprising cells that can be induced to differentiate into cell types that reflect lineages chosen from endodermal, ectodermal, and mesodermal embryonic germ layers.

The blastomere-derived pluripotent stem cells of the invention can also be induced to differentiate into specific cell types. In one embodiment, blastomere-derived pluripotent stem cells of the invention can be induced to differentiate into neural stem cells. As a nonlimiting example, a neural stem cell may be a cell that expresses Sox-1, Pax-6, and Nestin. In another embodiment, blastomere-derived pluripotent stem cells of the invention can be induced to differentiate into spontaneously beating cardiomyocytes characterized by contractile loci. In another embodiment, blastomere-derived pluripotent stem cells of the invention can be induced to differentiate into hepatocytes. As a nonlimiting example, a hepatocyte may be a cell that expresses alpha-fetal protein (AFP), albumin (ALB), transferrin (TFN), hepatic nuclear factor 4α (HNF4α), and C/EBPβ. A hepatocyte may also be a cell that expressed binding sites for the lectin, Dolichos biflorus agglutinin (DBA).

The blastomere-derived pluripotent stem cells of the invention can be used to produce cellular structures during in vitro culture that recapitulate the cellular structures that form during the course of normal embryonic development. In one embodiment, blastomere-derived pluripotent stem cells of the invention can give rise to a morula, as well as all of the embryonic structures that precede the morula stage of embryonic development. In another embodiment, blastomere-derived pluripotent stem cells of the invention can give rise, in vitro or in vivo, to a blastocyst, as well as all of the embryonic structures that precede the morula stage of embryonic development. In yet another embodiment, blastomere-derived pluripotent stem cells of the invention can be aggregated with ESC to form a chimeric embryo that can be brought to full-term using surrogate mothers. In still yet another embodiment, blastomere-derived pluripotent stem cells of the invention, without the introduction of additional ESC, can be used to produce an embryo that can be brought to full-term, using a surrogate mother. In one embodiment, the blastomere-derived pluripotent stem cells of the invention used to produce either chimeric or non-chimeric embryos are transgenic, wherein the transgene may be a transgene that causes the overexpression of a gene or a transgene that reduces the expression of a gene.

The blastomere-derived pluripotent stem cells of the invention may have therapeutic uses. In one embodiment, blastomere-derived pluripotent stem cells of the invention can be stored until needed for repairing damaged, diseased, or missing tissue. For example, tissues and organs that can be treated by the blastomere-derived pluripotent stem cells of the invention include bone, muscle, teeth, bladder, breast, brain, eye, adrenal gland, the cardiovascular system, small intestine, large intestine, kidney, liver, lung, pancreas, skin, stomach, and thyroid gland. In another embodiment, blastomere-derived pluripotent stem cells of the invention can be stored until needed as an autologous tissue source for repairing damaged, diseased, or missing tissue for the developed organism derived from the same preimplantation embryo from which the blastomere-derived pluripotent stem cells of the invention were derived.

### BRIEF DESCRIPTION OF THE FIGURES

FIG. 1: Schematic showing three possible protocols for establishing blastomere-derived pluripotent stem cells. Single blastomeres dissociated from two, four, and eight cell stage embryos and cultured in one of the three culture conditions shown: Condition A, Condition B, and Condition C.

FIG. 2: The development of single blastomeres into embryo-like cellular structures, grown in ES media and on MEF feeder layers. FIGS. 2A1-A7: Developmental stages recapitulated in vitro by ¼ blastomeres. Depicted are the 4 cell stage, 8 cell stage, morula, early blastula, and 3 stages of late blastula development. FIGS. 2B1-B7: Developmental stages recapitulated in vitro by ⅛ blastomeres. Depicted are the 4 cell stage, 8 cell stage, morula, early blastocyst, and 3 stages of late blastocyst development. FIGS. 2C1-C7: Developmental stages of control 4 cell embryos. Depicted are the 4 cell stage, 8 cell stage, morula, early blastula, and 1 stage of late blastula development.

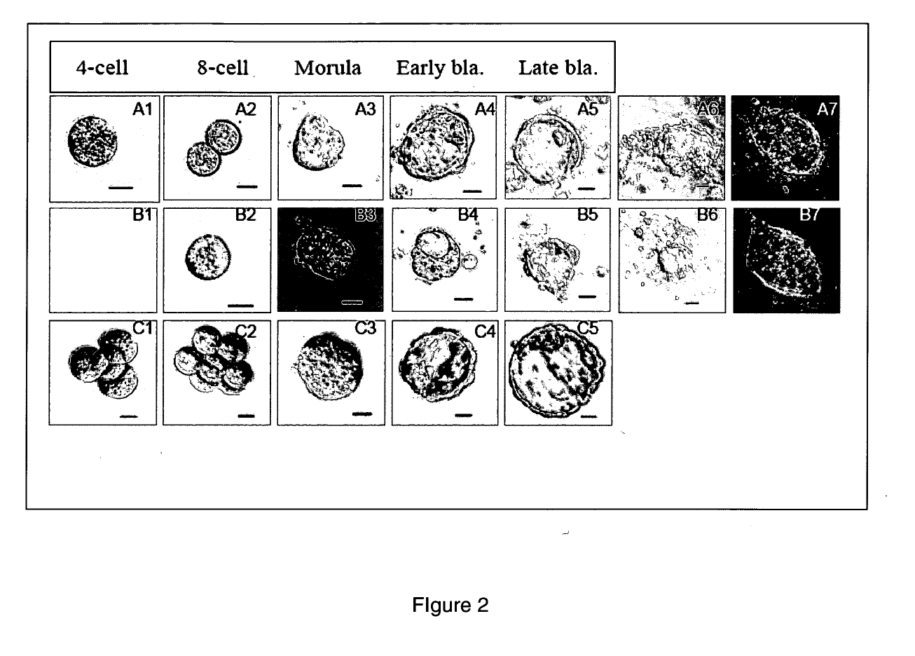

FIG. 3: Oct-4 expression by blastomere derivatives. Oct-4 protein expression was visualized by immunohistochemical analysis using FITC (green). Cell nuclei were visualized by DAPI staining (blue). FIGS. 3A1-A4: Expression of Oct-4 by ¼ blastomeres and derivatives, including a blastocyst-like cellular structure and intermediate structures. FIGS. 3B1-B3: Expression of Oct-4 by ⅛ blastomeres and derivatives, including a blastocyst-like cellular structure and intermediate structures. FIGS. 3C1-C4: Expression of Oct-4 by control embryo at the 4-cell, 8-cell, morula, and expanded blastocyst stages.

FIG. 4: Expression of pluripotent stem cell markers by cells derived from single, isolated blastomeres. FIG. 4A: RT-PCR expression analysis of pluripotent stem cell marker genes POUSF1, NANOG, FOXD3, REX1, SOX2, UTF1. Results are shown for the: embryonic stem cell line, D3; the single, isolated blastomere-derived pluripotent stem cell lines OF-1 and OE-1; and MEF cells, which served as a negative control. FIGS. 4B-E: Immunostaining analysis of OF-1 and OE-1 cell lines for Oct-4 and SSEA-1 expression.

FIG. 5: Karyotype analysis by GTG-banded metaphase spreads of single blastomere-derived pluripotent stem cell lines. FIG. 5A: Normal euploid 40-XY karyotype of the OF-1 cell line. FIG. 5B: Normal euploid 40-XX karyotype of the OE-31 cell line.

FIG. 6: Histological analysis of teratomas generated from single, isolated blastomere-derived pluripotent stem cell lines OF-1 (A-D) and OE-1 (E-H). FIG. 6A: OF-1-derived neural epithelium (ectoderm). FIG. 6B: OF-1-derived muscle tissue (mesoderm). FIG. 6C: OF-1-derived bone-like structure (mesoderm). FIG. 6D: OF-1-derived respiratory epithelium (endoderm). FIG. 6E: OE-1-derived skin-like tissue (ectoderm). FIG. 6F: OE-1-derived hair follicle-like structure (ectoderm). FIG. 6G: OE-1-derived cartilage tissue (mesoderm). FIG. 6H: OE-1-derived gastro-intestinal tract epithelium (endoderm). Bar: 20 μm.

FIG. 7: Expression of neural differentiation markers by OF-1 and OE-1 cell lines following in vitro neural differentiation. FIG. 7A: RT-PCR expression analysis of neural cell markers PAX6, MSL1, OLIGO2, and GFAP. Results are shown for the single, isolated blastomere-derived pluripotent stem cell lines OF-1 and OE-1 following in vitro neural differentiation. FIG. 7B: Immunofluorescence analysis of early neural cell markers Nestin and Pax6 by neural progenitors derived from single, isolated blastomere-derived pluripotent stem cell lines. FIG. 7C: Immunofluorescence analysis of early neural cell markers Nestin and Sox-1 by neural progenitors derived from single, isolated blastomere-derived pluripotent stem cell lines. FIG. 7D: Immunostaining analysis of mature neuronal and glial cell markers GFAP and microtubule-associated protein (MAP)2 by derivatives of neural progenitors derived from single, isolated blastomeres. FIG. 7E: Immunostaining analysis of mature neuronal and glial cell markers tyrosine hydroxylase (TH) and MAP2 by derivatives of neural progenitors derived from single, isolated blastomeres.

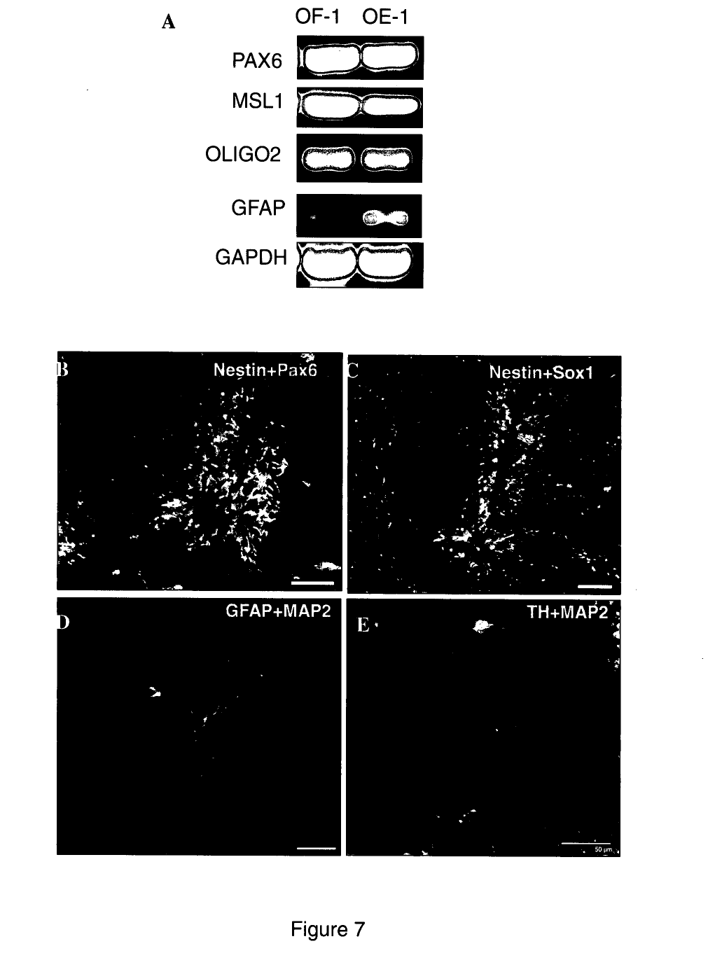

FIG. 8: Expression of cardiomyocyte differentiation markers by OF-1 and OE-1 cell lines following in vitro cardiomyocyte differentiation. FIG. 8A: RT-PCR expression analysis of cardiomyocyte markers: GATA-4, NCX-2.5, Mef-2C, NCX-1, cardiac troponin (cTn)-I, Anf, MLC-2V, and β-MHC. Results are shown for the single, isolated blastomere-derived pluripotent stem cell lines OF-1 and OE-1 following in vitro cardiomyocyte differentiation. FIG. 8B: Immunostaining analysis of α-actinin expression by cardiomyocytes derived from single, isolated blastomere-derived pluripotent stem cell lines. FIG. 8C: Immunostaining analysis of cTn-I expression by cardiomyocytes derived from single, isolated blastomere-derived pluripotent stem cell lines.

FIG. 9: Expression of hepatocyte differentiation markers by OF-1 and OE-1 cell lines following in vitro hepatocyte differentiation. FIG. 9A: RT-PCR expression analysis of hepatocyte cell markers albumin, α-fetoprotein, brachyury, HNF4α, Hex, Sox17, Fox2a, and GATA4. Results are shown for the single, isolated blastomere-derived pluripotent stem cell lines OF-1 and OE-1 following in vitro hepatocyte differentiation. FIG. 9B: Immunostaining analysis of α-fetoprotein expression by hepatocytes derived from single, isolated blastomere-derived pluripotent stem cell lines. FIG. 9C: Immunostaining analysis of transferrin expression by hepatocytes derived from single, isolated blastomere-derived pluripotent stem cell lines. FIG. 9D: Immunostaining analysis of albumin expression by hepatocytes derived from single, isolated blastomere-derived pluripotent stem cell lines. FIG. 9E: Immunostaining analysis of C/EBPβ expression by hepatocytes derived from single, isolated blastomere-derived pluripotent stem cell lines. FIG. 9F: Immunostaining analysis of HNFα expression by hepatocytes derived from single, isolated blastomere-derived pluripotent stem cell lines. FIG. 9G: Analysis of DBA reactivity by hepatocytes derived from single, isolated blastomere-derived pluripotent stem cell lines.

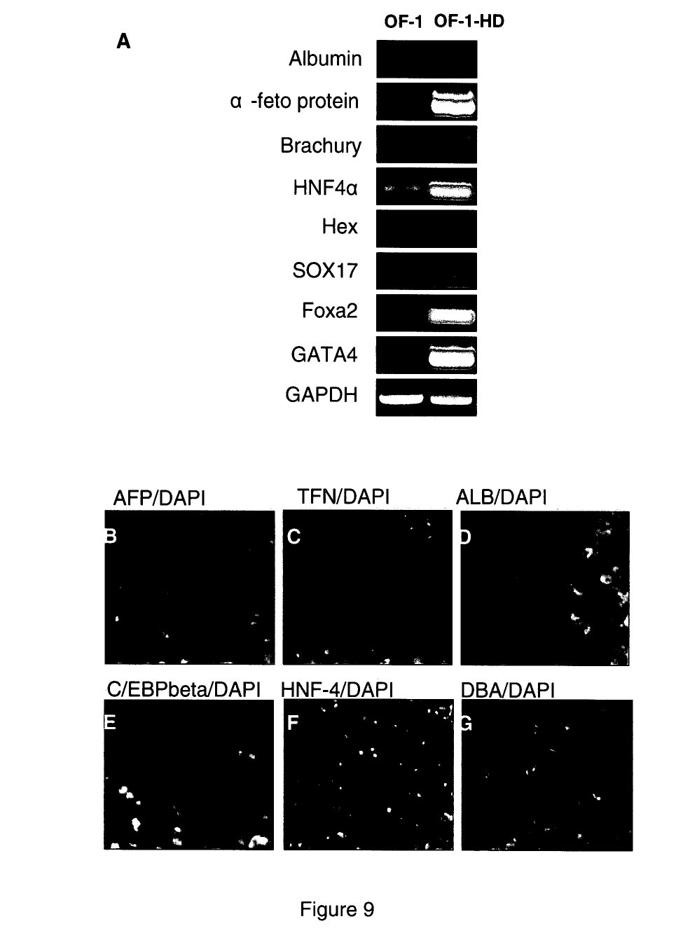

FIG. 10 Contribution of blastomere-derived pluripotent stem cells to chimeric mice. FIG. 10A: A female chimeric mouse showing contribution to fur color by a ¼ blastomere-derived pluripotent stem cell line (OF-1). FIG. 10B: A female chimeric mouse showing contribution to fur color by a ⅛ blastomere-derived pluripotent stem cell line (OE-5).

### BRIEF DESCRIPTION OF THE TABLES

Table 1: The DNA sequences of PCR primers used to characterize undifferentiated and differentiated blastomere-derived pluripotent stem cells.

Table 2: Antibodies used to characterize undifferentiated and differentiated blastomere-derived pluripotent stem cells.

Table 3: Total cell number, total number of OCT⁺ cells, and the OCT-4⁺ to OCT-4⁻ ratio of blastomere derivatives cultured in different conditions and derived from the ½, ¼, and ⅛ blastomeres.

### DETAILED DESCRIPTION OF THE INVENTION

The present invention is not limited to the particular methodology, protocols, cell lines, animal species or genera, and reagents described below. The terminology used to describe particular embodiments is not intended to limit the scope of the present invention, which will be limited only by the appended claims. As used herein, the singular forms “a,” “and,” and “the” include plural reference unless the context clearly dictates otherwise. Thus, for example, reference to “a cell” is a reference to one or more cells and includes equivalents thereof known to those skilled in the art.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices, and materials are now described. All publications and patents mentioned herein are hereby incorporated herein by reference for the purpose of describing and disclosing, for example, the constructs and methodologies that are described in the publications which might be used in connection with the presently described invention. The publications discussed above and throughout the text are provided solely for their disclosure prior to the effective date of the present application. Nothing herein is to be construed as an admission that the inventors are not entitled to antedate such disclosure by virtue of prior invention.

Definitions

A “derivative” of a cell or embryonic stem cell is a cell whose lineage can be traced.

A “differentiated cell” is a cell that has progressed down a developmental pathway, and includes lineage-committed progenitor cells and terminally differentiated cells.

The term “embryo” or “embryonic” as used herein includes a developing cell mass that has not implanted into the uterine membrane of a maternal host. Hence, the term “embryo” as used herein can refer to a fertilized oocyte, a cybrid, a pre-blastocyst stage developing cell mass, and/or any other developing cell mass that is at a stage of development prior to implantation into the uterine membrane of a maternal host. Embryos of the invention may not display a genital ridge. Hence, an “embryonic cell” is isolated from and/or has arisen from an embryo. An embryo can be representative of multiple stages of cell development. For example, a one cell embryo can be referred to as a zygote, a solid spherical mass of cells resulting from a cleaved embryo can be referred to as a morula, and an embryo having a blastocoel can be referred to as a blastocyst.

The term “embryonic stem cell” refers to pluripotent stem cells derived from an embryo in the blastocyst stage, or pluripotent cells produced by artificial means that have equivalent characteristics.

The term “embryoid bodies” refers to aggregates of differentiated and undifferentiated cells that appear when pluripotential stem cells overgrow in monolayer cultures, or are maintained in suspension cultures. Embryoid bodies are a mixture of different cell types, typically from several germ layers, distinguishable by morphological criteria and cell markers detectable by immunocytochemistry

“Feeder cells” or “feeders” are terms used to describe cells of one type that are co-cultured with cells of a second type, to provide an environment in which the cells of the second type can be maintained, and perhaps proliferate. The feeder cells can be from a different species than the cells they are supporting. For example, certain stem cells can be supported by mouse embryonic fibroblasts (from a primary culture or a telomerized line) or human fibroblast-like or mesenchymal cells. Typically (but not necessarily), feeder cells are inactivated by irradiation or treatment with an anti-mitotic agent such as mitomycin C, to prevent them from outgrowing the cells they are supporting.

The term “pluripotent” or “pluripotency” refers to cells with the ability to give rise to progeny that can undergo differentiation, under the appropriate conditions, into cell types that collectively demonstrate characteristics associated with cell lineages from all of the three germinal layers (endoderm, mesoderm, and ectoderm). Pluripotent stem cells can contribute to many or all tissues of a prenatal, postnatal or adult animal. A standard art-accepted test, such as the ability to form a teratoma in 8-12 week old SCID mice can be used to establish the pluripotency of a cell population. The term includes both established lines of stem cells and cells obtained from primary tissue that are pluripotent in the manner described. For the purposes of this disclosure, pluripotential cells are not embryonal carcinoma cells, and are not derived from a malignant source.

A cell “marker” is any phenotypic feature of a cell that can be used to characterize it or discriminate it from other cell types. A marker of this invention may be a protein (including secreted, cell surface, or internal proteins; either synthesized or taken up by the cell), a nucleic acid (such as an mRNA, or an enzymatically active nucleic acid molecule), or a polysaccharide. Included are determinants of any such cell components that are detectable by antibody, lectin, probe or nucleic acid amplification reaction that are specific for the cell type of interest. The markers can also be identified by a biochemical or enzyme assay that depends on the function of the gene product. Associated with each marker is the gene that encodes the transcript, and the events that lead to marker expression.

The term “stem cell” refers to a pluripotent or multipotent cell with the ability to self-renew, to remain undifferentiated, and to become differentiated into other cell types.

The term “transgene” broadly refers to any nucleic acid that is introduced into the genome of an animal, including but not limited to genes or DNA having sequences which are not normally present in the genome, genes which are present, but not normally transcribed and translated (“expressed”) in a given genome, or any other gene or DNA which one desires to introduce into the genome. This may include genes which may normally be present in the nontransgenic genome but which one desires to have altered in expression, or which one desires to introduce in an altered or variant form. The transgene may be specifically targeted to a defined genetic locus, may be randomly integrated within a chromosome, or it may be extrachromosomally replicating DNA. A transgene may include one or more transcriptional regulatory sequences and any other nucleic acid, such as introns, that may be necessary for optimal expression of a selected nucleic acid. A transgene can be coding or non-coding sequences, or a combination thereof. A transgene may comprise a regulatory element that is capable of driving the expression of one or more transgenes under appropriate conditions.

The term “transgenic cell” refers to a cell containing a transgene.

### EXAMPLES

The present invention is further illustrated by the following examples which should not be construed as limiting in any way.

### Example 1

### Embryo Recovery

Female C57BL/6 mice were superovulated by injection of 5 units of pregnant mare's serum gonadotropin (Sigma-Aldrich, St. Louis, Mo.-Aldrich, St. Louis, Mo.) followed 48 hours later by injection of 5 units of human chorionic gonadotropin (Sigma-Aldrich, St. Louis, Mo.). The superovulated females were then paired overnight with C57BL/6 males for mating. The following morning, females with vaginal plugs were selected for embryo collection. Typically, by this time post coitus (p.c.), most embryos could be expected to be at the 2 cell or 4 cell stages of development. Embryos at the 8 cell stage of development could be expected to have formed by 2.5 days p.c. Embryos were collected by flushing the oviduct with M2 medium (Sigma-Aldrich, St. Louis, Mo.). After briefly washing the embryos in M2 medium, the embryos were transferred into 35 mM non-adherent tissue culture plates that contained KSOM culture medium (Specialty Media, Phillipsburg, N.J.). The tissue culture plates were then kept in a tissue culture incubator set at 37° C. and 5% CO₂ for at least 2-3 minutes. Control, 8-cell stage embryos were cultured in KSOM culture medium until they reached the blastocyst stage. The embryos were then collected for ICM isolation and ESC derivation.

### Example 2

### Isolation and Culture of Single Blastomeres of Preimplantation Embryos

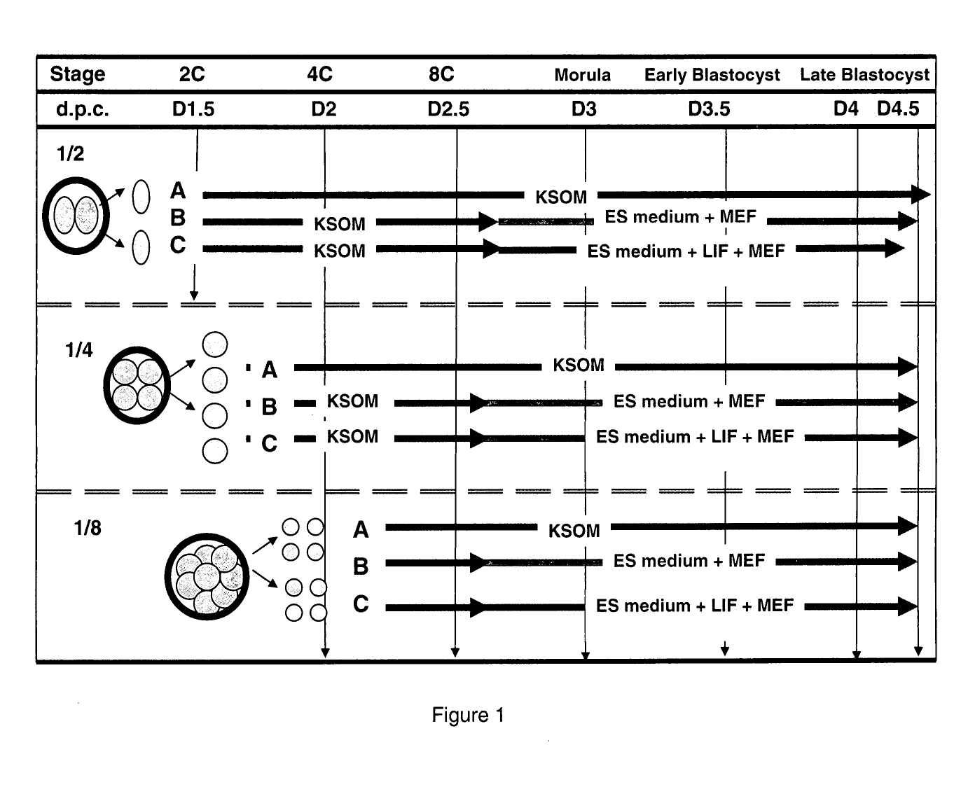

The zona pellucida of each 2 cell, 4 cell, or 8 cell embryo was removed by placing embryos into acidified (pH 2.5) Tyrode's medium (MediCult, Denmark) for 2-3 minutes at 37° C. followed by two brief washings in M2 medium (Sigma-Aldrich, St. Louis, Mo.). Zona-free embryos were then incubated in Ca²⁺ and Mg²⁺ free biopsy medium (MediCult, Denmark) for 10 minutes at 37° C. Individual blastomeres were isolated by repeatedly pipetting the zona-free embryos with a flame-polished glass micropipette. Subsequently, following a brief recovery in KSOM medium, isolated blastomeres were each transferred to 20 μl droplets of KSOM medium and incubated at 37° C. and 5% CO₂. Generally, within about 2.5 days, the blastomeres would rise to morula-like cell clusters, which were then cultured in one of three different culture conditions, designated conditions A, B, and C, respectively as depicted in FIG. 1. Culture condition A was defined by the continued culture of the blastomeres in the KSOM medium. Culture conditions B and C involved the dissociation of the morula-like cell clusters into single cell suspensions by tritiation (repeated pipetting). The cells were then co-cultured on a monolayer of mitomycin C-inactivated mouse embryo fibroblasts (MEFs) which were plated on gelatin coated tissue culture plates. MEFs were inactivated by adding 5 μg/ml mitomycin C (Sigma-Aldrich, St. Louis, Mo.) and incubating the cells for 30 minutes at 37° C. The medium used for condition B was ES culture medium (80% Dulbecco's modified Eagle medium supplemented with 20% fetal bovine serum, 0.1 mM β-mercaptoethanol, 1% nonessential amino acids, and 2 mM glutamine). Condition C was the same as condition B, except that the ESC culture medium was additionally supplemented with 1000 I.U./ml of leukemia inhibitory factor (LIF) (ESGRO, Chemicon, Billerica, Mass.) for the rest of culture period.

The pluripotency potential of ½, ¼, and ⅛ blastomere derivatives was determined under conditions A, B, and C. Unmanipulated embryos were used as controls. The percentage of the blastomeres that divided normally upon being cultured as single cells in KSOM medium prior to being allocated to condition A, B, or C was similar to that of the control embryos. For ½, ¼ and ⅛ blastomeres, respectively, the percentages of blastomeres that divided normally was 93%, 89% and 75%.

However, very few ¼ and ⅛ blastomeres continued to proliferate when cultured under Condition A. Consequently, no further efforts were made to characterize blastomere-derivatives produced under condition A. On the other hand, by two days (2.5 to 3 days p.c.) most blastomere derivatives that were transferred to Conditions B and C formed floating morula-like cell clusters. Within five to ten hours after forming, approximately half of the morula-like cell clusters attached to MEF feeder had subsequently flattened and developed into a structure with a colony morphology without demonstrating characteristics of blastocyst formation. The remaining morula-like cell clusters continued to develop into floating blastocyst-like structures.

Individual colonies cultured under conditions B and C were mechanically isolated from the MEF layers, titriated into single cells with 0.1% trypsin-EDTA solution (Invitrogen, Carlsbad, Calif., Carlsbad, Calif.), and replated onto fresh mitomycin C (Sigma-Aldrich, St. Louis, Mo.)-treated MEF feeder plated on gelatin-coated tissue culture plates in ESC culture medium. The ES cell culture medium comprised 80% Dulbecco's modified Eagle medium (DMEM, Invitrogen, Carlsbad, Calif.) supplemented with 20% fetal bovine serum (FBS; Hyclone, Logan, Utah), 0.1 mM β-mercaptoethanol (Sigma-Aldrich, St. Louis, Mo.), 1% nonessential amino acids (Invitrogen, Carlsbad, Calif.), and 2 mM glutamine (Invitrogen, Carlsbad, Calif.). After an initial outgrowth, the resulting colony was dissociated with 0.1% trypsin-EDTA solution (Invitrogen, Carlsbad, Calif.), pipetted, and plated onto new feeder plates for further expansion. After the first passage, only the colonies with ESC-like morphology were selected for further propagation. ESC-like colonies were split every two to three days by brief incubation in 0.1% trypsin-EDTA solution (Invitrogen, Carlsbad, Calif.) to disrupt cell-to-cell contacts followed by centrifugation to remove the trypsin solution. Cells were then replated onto dishes with new MEF feeder cells and fed with new ES medium daily. Cells were passaged until the majority of the blastomere-derived cells displayed ESC-like morphology. Typically, four to five days separated each passage and cells were generally passaged five to ten times. At the end of the final passage, the cells were prepared for cryopreservation and kept under liquid nitrogen storage conditions until needed for further characterization or experimentation.

### Example 3

### Oct-4 Expression by Blastomere Derivatives

Immunofluorescence analysis was used to assess expression of Oct-4 expression by blastomere derivatives. The expression of Oct-4 by a cell was considered to correlate with pluripotency. The protocol for immunofluorescence staining was performed as follows (Kuo H C et al. (2003) Biol Reprod 68:1727-35).

Blastomeres or their derivatives were plated onto cover slips coated with Matrigel (Invitrogen, Carlsbad, Calif.) and fixed with 4% paraformaldehyde for 20 minutes at room temperature. Blocking solution, which included PBS (Invitrogen, Carlsbad, Calif.), 0.1% BSA (Sigma-Aldrich, St. Louis, Mo.), 10% normal goat serum (Sigma-Aldrich, St. Louis, Mo.), and 0.2% Triton X-100 (Sigma-Aldrich, St. Louis, Mo.) was then added to the fixed cells. The fixed cells were incubated overnight with primary antibodies (Table 2) in PBS containing 0.1% BSA (Sigma-Aldrich, St. Louis, Mo.) and 10% normal goat serum (Sigma-Aldrich, St. Louis, Mo.) at 4° C. After three brief washes in PBS/0.1% BSA, an appropriate FITC-conjugated secondary antibody (Molecular Probes and Jackson ImmunoResearch) was chosen and then incubated with the cell preparations at 1:200 dilution for 30 minutes at room temperature. After three washes in PBS /0.1% BSA, cells were counterstained using a 300 nM solution of 49,6-diamindino-2-phenylindole dihydrochloride (Molecular Probes, Eugene, Oreg.). Coverslips were then mounted onto the slides with a glycerol-based mounting solution containing 2.5% polyvinyl alcohol and 1,4-diazabicyclo[2,2,2]octane (Sigma-Aldrich, St. Louis, Mo.). Images were captured by confocal microscopy.

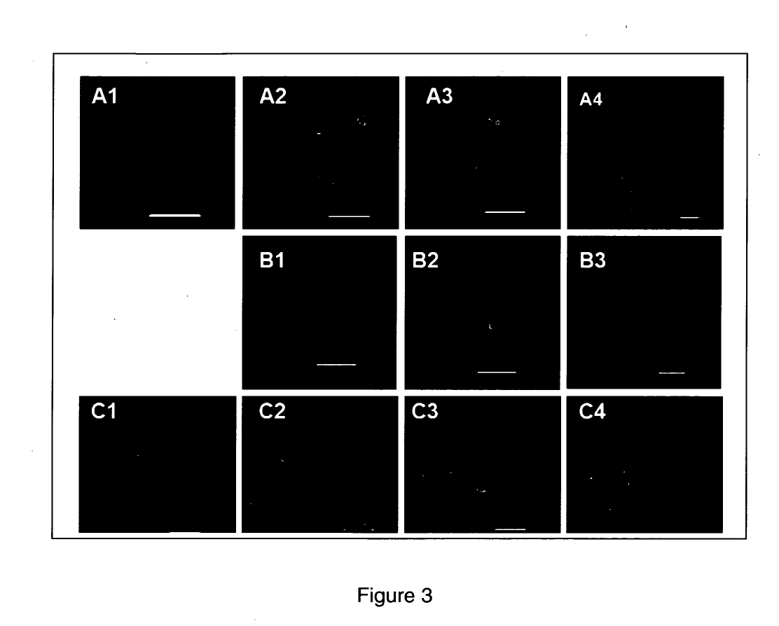

Immunofluorescence analysis of Oct-4 expression of blastomere derivatives performed on blastomere-derived cellular structures revealed that Oct-4 expression by blastomere derivatives was similar to that normally associated with control embryos. As observed during normal embryo development, Oct-4 was detectable at the transition from the 4-cell to the 8-cell stage (FIG. 3). Oct-4 was also found to be localized to the nucleus of cells of ½, ¼, and ⅛ derivatives at the 8-cell stage, morula stage, and the early blastocyst stage in the trophoectoderm (TE) and ICM (FIG. 3). In blastomere-derived structures that were equivalent to late blastocysts, intense Oct-4 signal was primarily associated with nuclei of the ICM and had diminished or disappeared in TE cells (FIG. 3).

The number of cells positive for Oct-4 expression (Oct-4⁺) under culture conditions A, B, and C was determined by counting the number of Oct-4⁺ cells as well as the total number of cells of each quantified blastocyst-like structure. Both the total cell number and number of Oct-4⁺ cells of the blastocyst-like structures derived from ½, ¼, and ⅛ blastomere were found to be significantly (P<0.05) reduced when cultured in the condition A. The highest number of total cells and Oct-4⁺ cells correlated with condition C. However, the addition of LIF into ES medium (condition C) had no statistically significant effect on total cell number or Oct-4⁺ cell number. Also, the ratio of Oct-4⁺ to Oct-4⁻ was significantly increased (P<0.05) in blastocyst-like structures derived from ¼ and ⅛ blastomere groups under condition C. Conversely, the total cell number, as well as the number of Oct-4⁺ cells, was significantly lower (P<0.05) when blastomere derivatives were cultured under condition A, rather than under conditions B or C.

### Example 4

### Characterization of Pluripotent Stem Cells

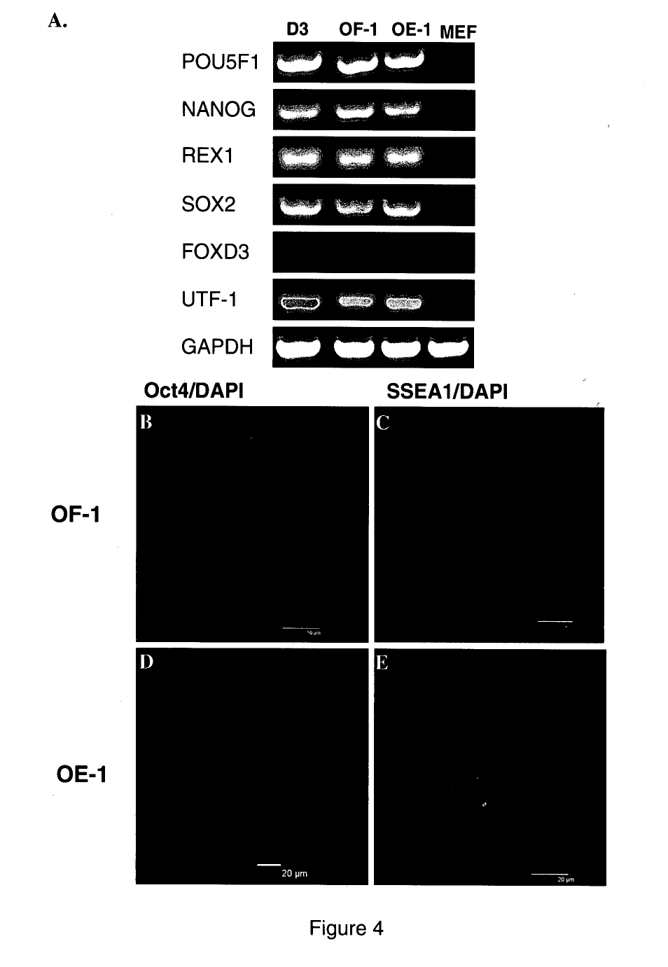

A total of 118 single blastomeres were isolated at an 86.8% isolation rate from thirty-four, 4-cell stage embryos over the course of three experiments. Similarly, a total of 425 single blastomeres were isolated at a 70.8% isolation rate from seventy-five 8-cell embryos over the course of seven experiments. Non-dividing derivatives of ¼ blastomeres, which represented 12/118, or 10.8% of the total number of initial blastomeres were excluded from further culture. Ultimately, three pluripotent stem cell lines were derived from ¼-blastomeres, which represented 2.8% of the initial number of single blastomeres used to generate these lines. These cell lines were designated as OF-1 to OF-3. Likewise, fifty-three pluripotent stem cell lines from ⅛-blastomeres were generated, which represented 12.5% of the initial number of single blastomeres. These cell lines were designated as OE-1 to OE-53. All established blastomere-derived pluripotent stem cell lines expressed key markers of undifferentiated pluripotent mouse stem cells, including SSEA-1, Oct-4, and alkaline phosphatase as detected by immunofluorescence analysis (FIGS. 4B-C). Oct-4 was detected as described in Example 3. The detection of SSEA-1 by biotinylated anti-SSEA-1 antibodies (Table 2) was performed by adding the antibody preparation to cells that were fixed as described in Example 3. The fixed cells were incubated overnight with primary antibodies (Table 1) in PBS containing 0.1% BSA (Sigma-Aldrich, St. Louis, Mo.) and 10% normal goat serum (Sigma-Aldrich, St. Louis, Mo.) at 4° C. After three brief washes in PBS/0.1% BSA, biotinylated secondary antibodies were linked to the biotin/avidin system (Vectastain; Vector Laboratories, Burlingame, Calif.) before signal amplification with 3′,3′-diaminobenzidine (DAB; Vector Laboratories) according to the manufacturer's protocols. After three washes in PBS (5 min each wash), cells were counterstained with 4′,6-diamindino-2-phenylindole dihydrochloride (DAPI; 300 nM; Molecular Probes, Inc., Eugene, Oreg.). Mounted slides were placed under a hood at room temperature (for biotin/avidin/DAB) before image capture with light microscopy.

The expression of alkaline phosphatase by the blastomere-derived pluripotent stem cells was detected following fixation of cells with 100% ethanol using a Vector blue kit (Vector Laboratories) according to the manufacturer's instructions.

Oct-4, Nanog, FoxD3, Rex-1, UTF-1 expression was determined by RT-PCR analysis (FIG. 4A). This analysis was performed as follows. Total RNA was isolated from cells using an RNAeasy extraction kit (Qiagen, Venlo, The Netherlands, Venlo, The Netherlands). To eliminate contaminating genomic DNA, 1 μg of total RNA was treated with 1 unit of DNase I (Invitrogen, Carlsbad, Calif.) for 15 min at 25° C., followed by inactivation of DNase I with 25 mM EDTA at pH 8.0 (Invitrogen, Carlsbad, Calif.) and incubation at 65° C. for 10 min. Reverse transcription and first strand cDNA synthesis was performed using SuperScript III One-Step RT-PCR kit (Invitrogen, Carlsbad, Calif.) according to the manufacturer's instruction. The first strand cDNA was further amplified by PCR using individual primer pairs for specific marker genes. The sequence, annealing temperature, and cycle number of each pair of primers are listed in Table 2. All PCR samples were analyzed by electrophoresis on 2% agarose gel containing 0.5 μg/ml ethidium bromide (Sigma-Aldrich, St. Louis, Mo.) All of the OF and OE lines expressed these genes.

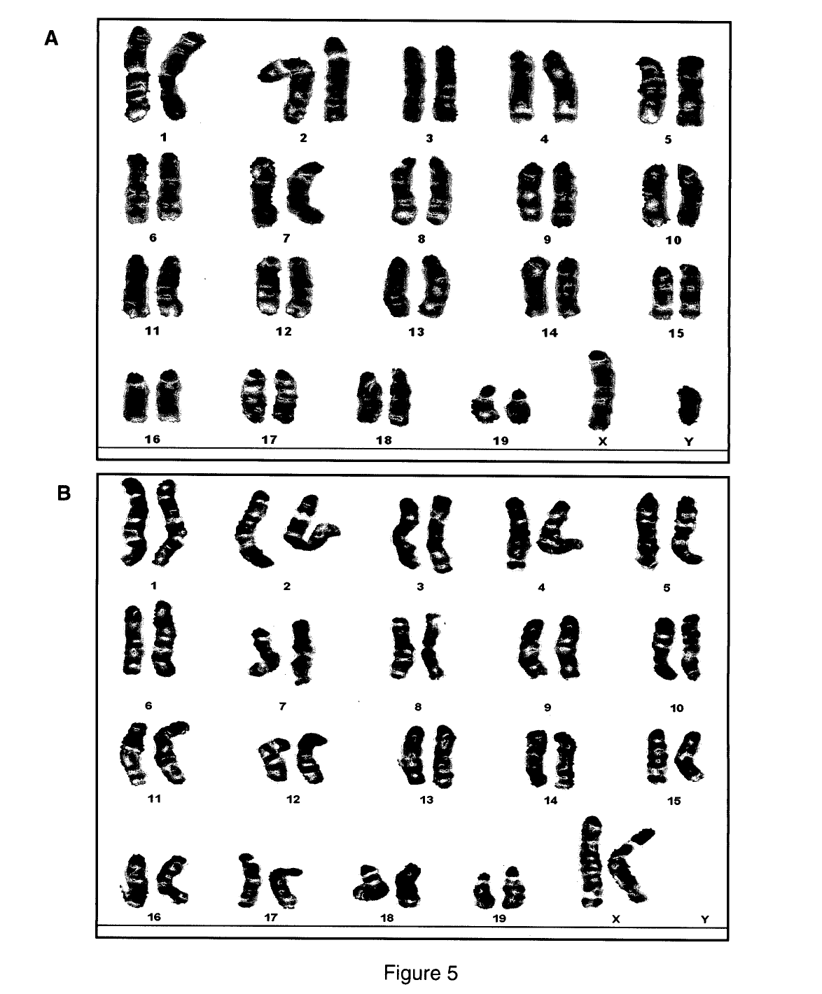

Karyotype analysis was performed on the OF and OE lines using a standard protocol. Briefly, the cells were centrifuged and the pellet was gently resuspended in 0.075 M KCl and incubated for 20 min at 37° C. followed by fixation with methanol:glacial acetic acid (3:1) and subjected to Giemsa staining for G-banding. Ten to fifteen of the separate metaphase spreads were examined from each culture. Karyotype analysis of the OF (OF1) and OE (4 lines: OE1-4) lines revealed that all of the lines tested have a normal complement of 40 chromosomes (FIG. 5).

### Example 5

### In Vivo Differentiation of Pluripotent Blastomere Derivatives

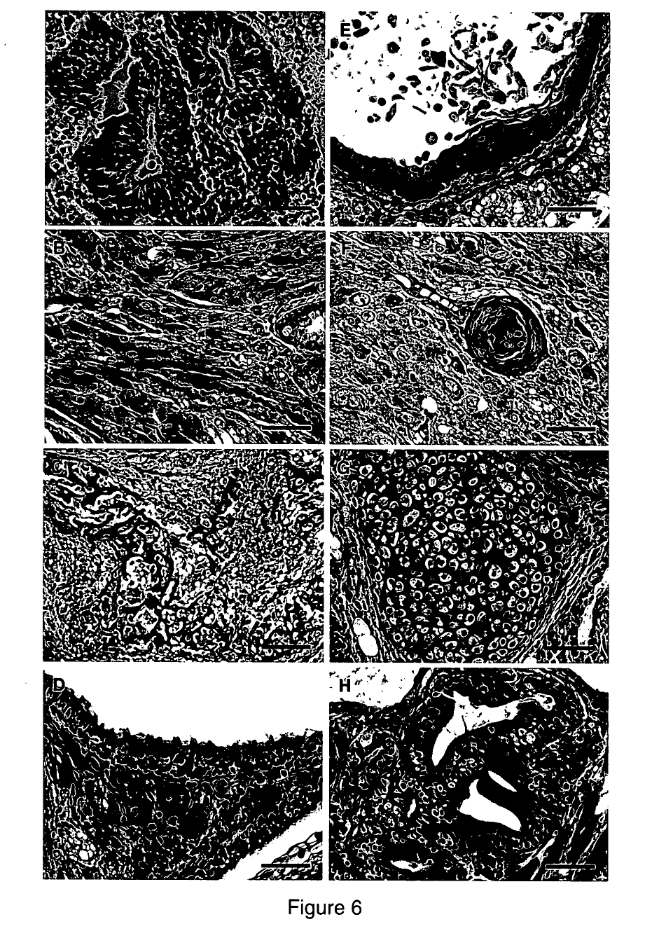

Potential of OF and OE cell lines to generate teratomas in vivo was investigated by the injection of six to eight week-old NOD-SCID mice into the rear leg muscles with OF or OE cells (5×10⁶ cells/site). Two OF lines and five OE lines were randomly selected for teratoma analysis. After tumors became palpable (approximately 5-7 weeks after injection) the tumors were excised and fixed overnight in 4% paraformaldehyde at 4° C. Paraffin sections were prepared and subjected to histological analysis by hematoxylin and eosin (H&E) staining. Histochemical analysis showed all the tested lines formed teratomas and contained derivatives of three embryonic germ layers, as demonstrated by ectoderm (neural epithelium and hair follicle-like cells), endoderm (striated muscle, bone, and cartilage), and endoderm (GI tract and respiratory epithelium) (FIG. 6).

### Example 6

### In Vitro Differentiation Potential of Pluripotent Blastomere Derivatives

The in vitro differentiation of the putative ES cells into ectodermal, mesodermal and endodermal lineages was based on well-established methods based on the formation of embryoid bodies (EB) (Kuo H C et al. (2003) Biol Reprod 68:1727-35). EB were formed by loosely detaching blastomere-derived stem cells from MEF feeder cells by a 10-20 minute incubation of the co-culture with 1 mg/ml of collagenase IV at 37° C. The blastomere-derived cells were then carefully aspirated into a micropipette, rinsed in ES medium three times, and cultured in suspension in ultra low attachment dishes (Corning Life Sciences) and ES medium to generate EBs.

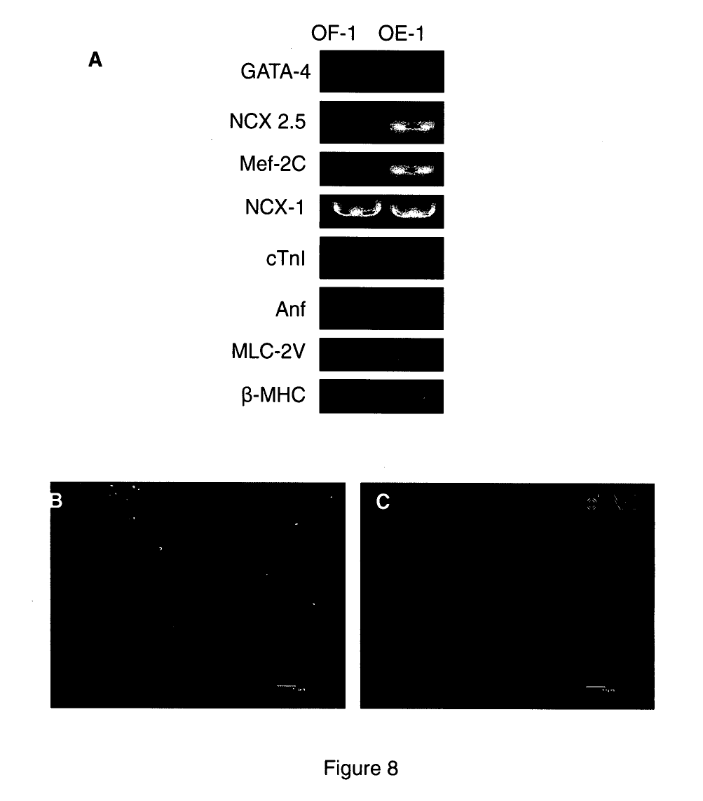

Neural differentiation of blastomere derivatives demonstrated the capacity of the blastomere derivatives to differentiate into an ectodermal cell lineage and was performed in two basic steps. First, blastomere-derived stem sells were differentiated into neuronal progenitor cells. Then, the neuronal progenitor cells were allowed to differentiate into mature neurons and glial cells. Neuronal progenitors were produced by allowing EBs to attach and grow on gelatin-coated culture dishes in ES medium following 4 days in suspension culture. ES medium was replaced with serum-free N2 medium, which was composed of a 1:1 mixture of DMEM and F12 medias (Invitrogen, Carlsbad, Calif.) supplemented with 10 ng/ml FGF2 (R&D Systems, Minneapolis, Minn.), and 1% N2 supplement (Invitrogen, Carlsbad, Calif.). The medium was changed every 2 days, whereas FGF2 was added daily. Rosette-like structures found in the resulting cell colonies represented early neural epithelium (neuronal progenitors). These cells were isolated from the culture and plated onto Matrigel (Invitrogen, Carlsbad, Calif.) coated coverslips and allowed to continue to differentiate in N3 medium (N2 medium supplemented with 1X B27 supplement, Invitrogen, Carlsbad, Calif.). Characterization of neural cells was by RT-PCR (FIG. 8A) was performed according to the general protocol described in Example 4 in conjunction with primer sets designed to amplify the following cardiomyocyte-specific markers found in Table 1: PAX6, MSL1, OLIGO2, and GFAP. Immunofluorescence analysis of early neural cell markers Nestin, Pax6, and Sox-1 (7B-C) was performed according to the general protocol found in Example 3. Similarly, immunofluorescence analysis demonstrated the expression of mature neuronal markers microtubule-associated protein (MAP)2 and tyrosine hydroxylase (TH) as well as glial cell marker GFAP.

Cardiac differentiation of blastomere derivatives demonstrated the capacity of the blastomere derivatives to differentiate into a mesodermal cell lineage. Differentiation was accomplished by culturing EBs in differentiation medium (DM), which was composed of 90% DMEM (Invitrogen, Carlsbad, Calif.) and 10% FBS (Hyclone, Logan, Utah) for 8 days in suspension. EBs were then plated in DM containing 10⁻⁹ M 5-aza-2′deoxycytidine (Sigma-Aldrich, St. Louis, Mo.) and 10₋₄ M ascorbic acid (Sigma-Aldrich, St. Louis, Mo.) for 4 days. Finally, the cells were cultured in DM for 20 days. Beating foci of contracting cells were mechanically isolated from EB outgrowths by using 26G syringe needles. The long-term culture of the EBs derived from OF and OE lines in cardiac-differentiation medium resulted in more than 85% (n=573; five independent experiments) of the EBs becoming positive for spontaneously contracting loci. The cardiomyocyte phenotype of the contracting cells isolated from the beating areas was confirmed by detection of contractile/sarcomeric protein expression (FIG. 8A) and cardiomyocyte-relevant genes (FIG. 8A). Characterization of the beating cells by RT-PCR (FIG. 8A) was performed according to the general protocol described in Example 4 in conjunction with primer sets designed to amplify the following cardiomyocyte-specific markers found in Table 1: GATA-4, NCX-2.5, Mef-2C, NCX-1, cardiac troponin (cTn)-I, Anf, MLC-2V, and P-MHC. Immunofluorescence analysis of cardiomyocyte-specific markers (FIGS. 8B-8C) was performed according to the general protocol found in Example 3 in conjunction with the following cardiomyocyte-specific antibodies found in Table 2: (α-actinin and cTn-I.

Hepatic differentiation of blastomere derivatives was performed by culturing the cells in ES medium and adding differentiation factors in a step-wise manner in accordance with the following protocol. After six days of culture, 20 ng/ml recombinant human acidic fibroblast growth factor (aFGF) (R&D Systems, Minneapolis, Minn.) and 10 ng/ml recombinant human basic fibroblast growth factor (bFGF) (R&D Systems, Minneapolis, Minn.) were added to the EBs in order to initiate hepatic differentiation. At day 8, 10 ng/ml rat recombinant hepatocyte growth factor (R&D Systems, Minneapolis, Minn.) was added as a midstage factor for expansion of hepatic progenitors. Then 10 ng/ml recombinant human oncostatin M (R&D Systems, Minneapolis, Minn.), 10⁻⁷ M dexamethasone (Sigma-Aldrich, St. Louis, Mo.), and ITS (insulin 10 μg/ml, transferrin 5 μg/ml, selenium 5 ng/ml, Invitrogen, Carlsbad, Calif.) were added at day 14. Cells were then cultured for 3 additional days to induce the maturation of hepatocytes, which are representative of an endodermal cell type.

### Example 7

### Contribution of Blastomere Derivatives to the Development of Chimeric Animals

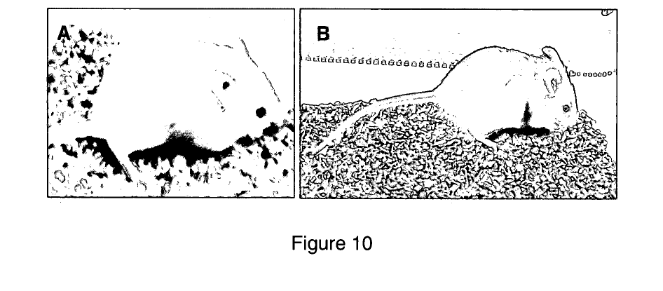

In order to produce the chimeras, wild-type blastocysts were first collected from the uteri of superovulated females of either CD-1 or DCB strain by flushing with M2 medium (Sigma-Aldrich, St. Louis, Mo.). Then, ten to twenty single OF-1 or OE-5 cells, which had been transfected with pCCALL-2, a Lac-Z plasmid expression construct, were microinjected into 3.5 days old blastocysts of FVB or CD-1 mice. The resulting chimeric embryos (10-15 embryos/host) were transferred into E2.5 pseudopregnant females (foster mother per line) and carried to term. To confirm the chimerism in the offspring, genomic DNA was extracted from tail biopsies and subject to PCR analysis using eGFP or LacZ specific primers. Subsequently, the male offspring showing chimerism in coat color were mated to their females counterparts to test for germ-line transmission. Eight pups were born from OF-1 line and four of them were chimeras as judged by their coat color (FIG. 10A). One of three pups born from line OE-1 was chimeric (FIG. 10B).

TABLE 1

DNA sequences of PCR primers used for cell characterizations.

| | | | | Ann. | Product |
|---|---|---|---|---|---|
| Cell | | | | temp | size |
| Type | Gene | Sequence (5′–3′) | Antisense(5′–3′) | (° C.) | (bp) |
| ES | Oct3/4 | CTGAGGGCCAGGCAGGAGCACGAG | CTGTAGGGAGGGCTTCGGGCACTT | 55 | 485 |
| | Nanog | AGGGTCTGCTACTGAGATGCTCTG | CAACCACTGGTTTTTCTGCCACCG | 55 | 364 |
| | REX-1 | GGCCAGTCCAGAATACCAGA | GAACTCGCTTCCAGAACCTG | 55 | 231 |
| | SOX2 | GTGGAAACTTTTGTCCGAGAC | TGGAGTGGGAGGAAGAGGTAAC | 55 | 602 |
| | FOXD3 | TCTTACATCGCGCTCATCAC | TCTTGACGAAGCAGTCGTTG | 55 | 172 |
| | UTF-1 | TGGAGTGCTCGAGAGACGGA | ACGTCCAGGGCCAGTAGAGC | 55 | 306 |
| | GAPDH | ACCACAGTCCATGCCATCAC | TCCACCACCCTGTTGCTGTA | 55 | 452 |
| Neural | PAX6 | CCATCTTTGCTTGGGAAATCCG | GCTTCATCCGAGTCTTCTCCGTTAG | 55 | 312 |
| | MSI1 | CGAGCTCGACTCCAAAACAAT | GGCTTTCTTGCATTCCACCA | 55 | 303 |
| | VIM | GCCATCAACACTGAGTTCAA | CTCCTCCTGCAATTTCTCTC | 55 | 306 |
| | GFAP | CACCCTGAGGCAGAAGCTCCAA | GCCACATCCATCTCCACGTG | 55 | 234 |
| | Oligo2 | TGCGAAGCTCTTTGTTCACG | ACGTTGTAATGCAGGTCGCG | 55 | 155 |
| Cardiac | GATA-4 | CACTATGGGCACAGCAGCTCC | TTGGAGCTGGCCTGCGATGTC | 55 | 140 |
| | NCX2.5 | CAGTGGAGCTGGACAAAGCC | TAGCGACGGTTCTGGAACCA | 55 | 217 |
| | Mef-2C | AGATACCCACAACACACCACG | ATCCTTCAGAGAGTCGCATGCGCTT | 55 | 167 |
| | NCX-1 | AGGAAAAGAGATGTATGGCC | GGCTGCTTGTCATCATATTC | 55 | 108 |
| | cTnI | AGATTGCAGATCTGACCCAG | CCTCAGGTCCAAGGATTCCT | 55 | 140 |
| | Anf | TCTTCCTCGTCTTGGCCTTT | CAGAGAAAGGGGACGTCTCA | 60 | 173 |
| | MLC-2v | TGCCCTAGGACGAGTGAACG | AGCCTTCAGTGACCCTTTGC | 60 | 187 |
| | β-MHC | GCCAACACCAACCTGTCCAAGTTC | CTGCTGGAGAGGTTATTCCTCG | 55 | 180 |
| Hepatic | GAPDH | AAGGTCGGTGTGAACGGATT | TGGTGGTGCAGGATGCATTG | 58 | 450 |
| | Albumin | GCTACGGCACAGTGCTTG | CAGGATTGCAGACAGATAGTC | 58 | 266 |
| | AFP | GCTCACACCAAAGCGTCAAC | CCTGTGAACTCTGGTATCAG | 58 | 410 |
| | Brachury | CATGTACTCTTTCTTGCTGG | GGTCTCGGGAAAGCAGTGGC | 58 | 312 |
| | HNF4α | ACACGTCCCCATCTGAAGGTG | CTTCCTTCTTCATGCCAGCCC | 58 | 269 |
| | Hex | TTCCCGCGGACGGTGAACGAC | TCATCCAGCATTAAAGTAGCCTTT | 58 | 540 |
| | SOX17 | GCCAAAGACGAACGCAAGCGGT | TCATGCGCTTCACCTGCTTG | 58 | 228 |
| | FOXA2 | TGGTCACTGGGGACAAGGGAA | GCAACAACAGCAATAGAGAAC | 58 | 289 |
| | GATA4 | ATGTATCAGAGCTTGGCCAT | CAGGAATCTGAGGAGGGAA | 58 | 397 |

TABLE 2
Antibodies used for cell characterizations.

| Antibodies | Dilution | Isotype | Source |
|---|---|---|---|
| Troponin 1 | 1:200 | IgG | Item No. FL-210, Santa Cruz Biotechnology, |
| (cTn1) | | | Inc. (Santa Cruz, CA) |
| α-Actinin | 1:800 | IgG | Item No. A-7811, Sigma-Aldrich, St. Louis, MO |
| (Sarcomeric) | | | |
| C/EBPβ | 1:75 | IgG | Santa Cruz Biotechnology, Inc. (Santa Cruz, |
| | | | CA) |
| HNF4α | 1:100 | IgG | Santa Cruz Biotechnology, Inc. (Santa Cruz, |
| | | | CA) |
| transferrin | 1:200 | IgG | Dako (Glostrup, Denmark) |
| Alpha fetal | 1:200 | IgG | Dako (Glostrup, Denmark) |
| protein | | | |
| DBA | 1:500 | IgG | Vector Laboratories (Burlingame, CA) |
| albumin | 1:300 | IgG | Vector Laboratories (Burlingame, CA) |
| Fluorescein | 1:300 | IgG | Vector Laboratories (Burlingame, CA) |
| streptavidin | | | |
| Oct-3/4 | 1:200 | IgG | Item No. sc-5279, Santa Cruz Biotechnology, |
| | | | Inc. (Santa Cruz, CA) |
| SSEA1 | 1:30 | IgM | Item No. MAB4301, Chemicon, Billerica, MA |
| GFAP | 1:200 | IgG | Item No. MAB3402, Chemicon, (Billerica, MA) |
| Nestin | 1:1000 | IgG | Item No. MAB353, Chemicon (Billerica, MA) |
| Sox1 | 1:100 | Purified | Item No. AB5768, Chemicon (Billerica, MA) |
| | | immunoglobin | |
| Pax6 | 1:100 | IgG | Item No. AB5409, Chemicon (Billerica, MA) |
| MAP2 | 1:1000 | Purified | Item No. AB5622, Chemicon (Billerica, MA) |
| | | immunoglobin | |
| TH | 1:200 | IgG | Item No. MAB318, Chemicon (Billerica, MA) |

TABLE 3
Analysis of OCT expression

| Developmental Stage of | OCT-4⁺ | OCT-4⁻ | Total | OCT-4⁺/OCT-4⁻ |
|---|---|---|---|---|
| Blastomere Donor Embryo and | Cell Number | Cell Number | Cell Number | ratio |
| Culture Conditions (A, B, or C) | (mean ± SEM) | (mean ± SEM) | (mean ± SEM) | (mean ± SEM) |
| 2-cell (A) (N = 33) | 2.76 ± 0.35 | 32.90 ± 1.39 | 35.46 ± 1.48 | 0.09 ± 0.01 |
| 2-cell (B) (N = 14) | 6.00 ± 1.20 | 43.79 ± 3.97 | 49.79 ± 4.61 | 0.14 ± 0.03 |
| 2-cell (C) (N = 13) | 12.08 ± 2.60 | 62.93 ± 5.86 | 75.00 ± 7.84 | 0.18 ± 0.03 |
| 4-cell (A) ND | ND | ND | ND | ND |
| 4-cell (B) (N = 8) | 6.25 ± 1.31 | 55.75 ± 6.80 | 62.00 ± 7.95 | 0.11 ± 0.02 |
| 4-cell (C) (N = 19) | 10.58 ± 1.64 | 52.63 ± 3.47 | 63.21 ± 3.96 | 0.21 ± 0.03 |
| 8-cell (A) ND | ND | ND | ND | ND |
| 8-cell (B) (N = 11) | 2.36 ± 1.16 | 32.55 ± 5.40 | 34.91 ± 6.24 | 0.07 ± 0.08 |
| 8-cell (C) (N = 19) | 5.05 ± 1.27 | 41.90 ± 3.97 | 46.95 ± 4.69 | 0.11 ± 0.03 |
| Control (A) | ND | ND | ND | ND |
| Control (B) (N = 9) | 16.33 ± 2.64 | 76.11 ± 6.86 | 92.44 ± 8.61 | 0.22 ± 0.03 |
| Control (C) (N = 8) | 50.75 ± 13.30 | 139.88 ± 24.88 | 190.63 ± 36.80 | 0.34 ± 0.05 |

ND: not determined.

What is claimed:

1. A method of producing pluripotent mammalian stem cells from a single blastomere cell comprising the steps of:
dissociating blastomeres from preimplantation embryos; and
culturing at least one blastomere to give rise to pluripotent stem cells.

2. The method of claim 1, wherein the at least one blastomere is isolated from an embryo at the two-cell stage.

3. The method of claim 1, wherein the at least one blastomere is isolated from an embryo at the four-cell stage.

4. The method of claim 1, wherein the at least one blastomere is isolated from an embryo at the eight-cell stage.

5. The method of claim 1, wherein the at least one blastomere is transgenic.

6. The method of claim 1, wherein the at least one blastomere is derived from a mouse embryo.

7. The method of claim 1, wherein the at least one blastomere is derived from a human embryo.

8. The method of claim 1, wherein the at least one blastomere is derived from a non-human primate embryo.

9. The method of claim 1, wherein the at least one blastomere is derived from a cow embryo.

10. The method of claim 1, wherein the at least one blastomere is derived from a sheep embryo.

11. The method of claim 1, wherein the at least one blastomere is derived from a goat embryo.

12. The method of claim 1, wherein the at least one blastomere is derived from a pig embryo.

13. A pluripotent stem cell produced according to the method of claim 1.

14. The pluripotent stem cell of claim 13, wherein the pluripotent stem cell maintains a stable, euploid chromosome karyotype.

15. The pluripotent stem cell of claim 13, wherein the pluripotent stem cell can give rise to cell types chosen from ectodermal, endodermal, and mesodermal cell lineages.

16. The pluripotent stem cell of claim 13, wherein stem cell derivatives of the pluripotent stem cell can be cultured in vitro for a period of at least one year without experiencing a loss in pluripotency.

17. The pluripotent stem cell of claim 13, wherein the pluripotent stem cell expresses at least one cell marker chosen from Oct-4, Nanog, Sox2, FoxD3, SSEA-1, and alkaline phosphatase (AP).

18. The pluripotent stem cell of claim 13, wherein the pluripotent stem cell is transgenic.

19. The pluripotent stem cell of claim 13, wherein the pluripotent stem cell is used for gene therapy.

20. The pluripotent stem cell of claim 13, wherein the pluripotent stem cell is used as part of therapy to treat a human disease.

21. The human disease of claim 20, wherein the human disease is chosen from cardiovascular diseases, neurological diseases, reproductive diseases, cancers, eye diseases, endocrine diseases, pulmonary diseases, metabolic diseases, hereditary diseases, autoimmune disorders, and aging.

22. The pluripotent stem cell of claim 12, wherein the pluripotent stem cell is used as part of a therapy to regenerate human tissue.

23. The human tissue of claim 22, wherein the lineage of the regenerated tissue is chosen from bone, muscle, teeth, bladder, breast, brain, eye, adrenal gland, the cardiovascular system, small intestine, large intestine, kidney, liver, lung, pancreas, skin, stomach, and thyroid gland
